(19) United States
(12) Patent Application Publication
(10) Pub. No.: US 2022/0401209 A1
(43) Pub. Date: Dec. 22, 2022

(54) SUPRA AORTIC ACCESS TRIFURCATED MODULAR STENT ASSEMBLY AND METHOD

(71) Applicant: Medtronic Vascular, Inc., Santa Rosa, CA (US)

(72) Inventors: Keith PERKINS, Santa Rosa, CA (US); Mark STIGER, Santa Rosa, CA (US); Steven CLAESSENS, Santa Rosa, CA (US); Travis ROWE, Santa Rosa, CA (US); Mark YOUNG, Santa Rosa, CA (US)

(21) Appl. No.: 17/894,619

(22) Filed: Aug. 24, 2022

Related U.S. Application Data
(62) Division of application No. 16/585,768, filed on Sep. 27, 2019.

(51) Int. Cl. A61F 2/07; A61F 2/24

(52) U.S. Cl. CPC A61F 2/07; A61F 2/24; A61F 2002/067

(57) ABSTRACT

The techniques of this disclosure generally relate to an assembly including a trifurcated modular stent device. The trifurcated modular stent device includes a main body, a bypass gate extending distally from a distal end of the main body, a primary artery leg extending distally from the distal end of the main body, and a distal artery leg extending distally from the distal end of the main body. The trifurcated modular stent device is delivered via supra aortic access such that the primary artery leg is deployed within the brachiocephalic artery providing immediate perfusion thereof.

### CROSS-REFERENCE TO RELATED APPLICATIONS

This application is a divisional of U.S. patent application Ser. No. 16/585,768 filed on Sep. 27, 2019, entitled “SUPRA AORTIC ACCESS TRIFURCATED MODULAR STENT ASSEMBLY AND METHOD” of Perkins et al., which is incorporated herein by reference in its entirety.

### FIELD

The present technology is generally related to an intra-vascular device and method. More particularly, the present application relates to a device for treatment of intra-vascular diseases.

### BACKGROUND

Aneurysms, dissections, penetrating ulcers, intramural hematomas and/or transections may occur in blood vessels, and most typically occur in the aorta and peripheral arteries. The diseased region of the aorta may extend into areas having vessel bifurcations or segments of the aorta from which smaller “branch” arteries extend.

The diseased region of the aorta can be bypassed by use of a stent-graft placed inside the vessel spanning the diseased portion of the aorta, to seal off the diseased portion from further exposure to blood flowing through the aorta.

The use of stent-grafts to internally bypass the diseased portion of the aorta is not without challenges. In particular, care must be taken so that critical branch arteries are not covered or occluded by the stent-graft yet the stent-graft must seal against the aorta wall and provide a flow conduit for blood to flow past the diseased portion.

### SUMMARY

The techniques of this disclosure generally relate to an assembly including a trifurcated modular stent device. The trifurcated modular stent device includes a main body, a bypass gate extending distally from a distal end of the main body, a primary artery leg extending distally from the distal end of the main body, and a distal artery leg extending distally from the distal end of the main body. The trifurcated modular stent device is delivered via supra aortic access such that the primary artery leg is deployed within the brachiocephalic artery providing immediate perfusion thereof.

In one aspect, the present disclosure provides an assembly including a trifurcated modular stent device. The trifurcated modular stent device includes a main body configured to be located in an aorta, a bypass gate configured to be located in the aorta, a primary artery leg configured to be located within a brachiocephalic artery, and a distal artery leg configured to perfuse a distal artery distal of the brachiocephalic artery.

In another aspect, the present disclosure provides a method including introducing a delivery system including a trifurcated modular stent device via supra aortic access through a brachiocephalic artery. The delivery system is advanced into an aorta. The trifurcated modular stent device is deployed from the delivery system such that a main body of the trifurcated modular stent device engages the aorta, a primary artery leg of the trifurcated modular stent device engages the brachiocephalic artery, a bypass gate of the trifurcated modular stent device engages the aorta, and a distal artery leg of the trifurcated modular stent device is located within the aorta proximal of a distal artery distal of the brachiocephalic artery.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a perspective view of a trifurcated modular stent device in accordance with one embodiment.

FIG. 2 is an exploded perspective view of the trifurcated modular stent device of FIG. 1 in accordance with one embodiment.

FIG. 3 is a distal end plan view of the trifurcated modular stent device along the line III of FIG. 1 in accordance with one embodiment.

FIG. 4 is a cross-sectional view of a vessel assembly including the trifurcated modular stent device of FIGS. 1, 2, and 3 after deployment in accordance with one embodiment.

FIG. 5 is a cross-sectional view of the vessel assembly of FIG. 4 at a later stage after deployment of a bridging stent graft in accordance with one embodiment.

FIG. 6 is a cross-sectional view of the vessel assembly of FIG. 5 at a final stage after deployment of a tube graft and a proximal cuff into the trifurcated modular stent device in accordance with one embodiment.

FIG. 7 is a cross-sectional view of the vessel assembly of FIG. 4 at a later stage after deployment of a bridging stent graft in accordance with another embodiment.

### DETAILED DESCRIPTION

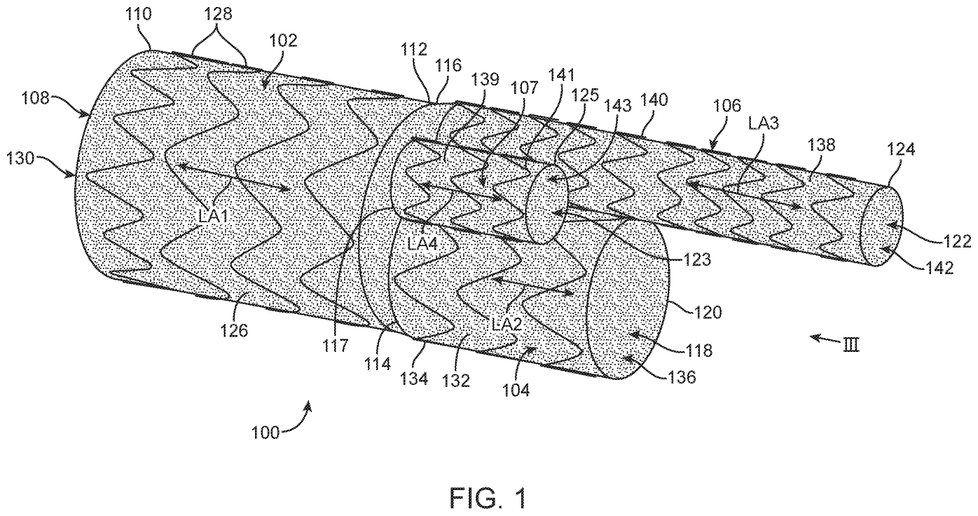

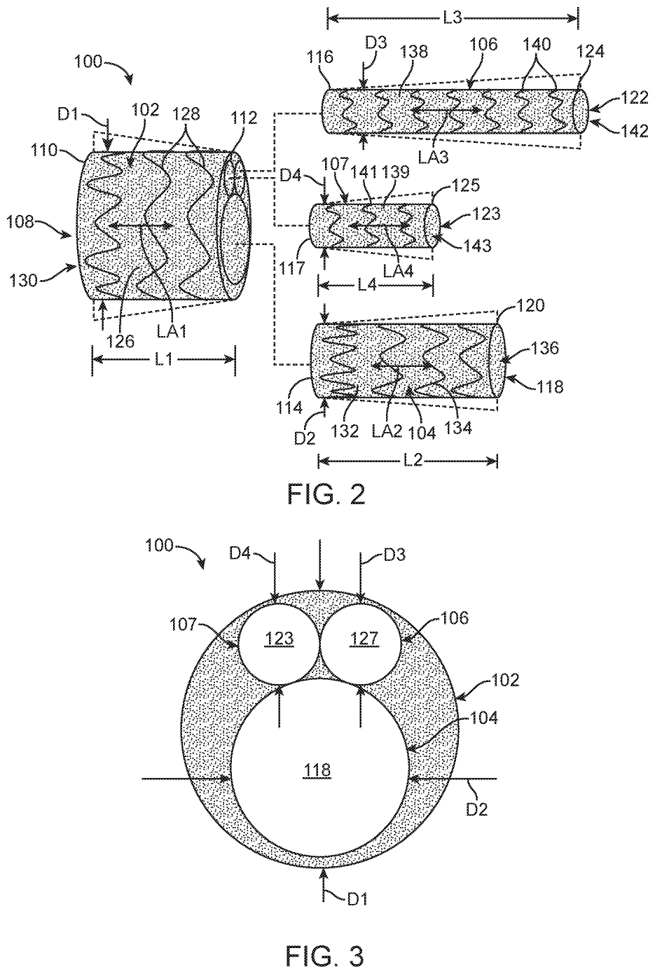

Now in more detail, FIG. 1 is a perspective view of a trifurcated modular stent device 100 in accordance with one embodiment. FIG. 2 is an exploded perspective view of trifurcated modular stent device 100 of FIG. 1 in accordance with one embodiment. FIG. 3 is a distal end plan view of trifurcated modular stent device 100 along the line III of FIG. 1 in accordance with one embodiment. In FIG. 2, trifurcated modular stent device 100 is exploded to illustrate the various features thereof. However, in light of this disclosure, those of skill in the art will understand that trifurcated modular stent device 100 is a unitary piece.

Referring now to FIGS. 1, 2, and 3 together, trifurcated modular stent device 100, sometimes called a prosthesis or aortic arch prosthesis, includes a main body 102, a bypass gate 104, a primary artery leg 106, sometimes called a brachiocephalic artery (BCA) leg/limb 106, and a distal artery leg 107.

In accordance with this embodiment, main body 102 includes a main body proximal opening 108 at a proximal end 110 of main body 102. A distal end 112 of main body 102 is coupled to a proximal end 114 of bypass gate 104, a proximal end 116 of primary artery leg 106, and a proximal end 117 of distal artery leg 107.

Bypass gate 104 includes a distal opening 118 at a distal end 120 of bypass gate 104. Primary artery leg 106 includes a distal opening 122 at a distal end 124 of primary artery leg 106. Distal artery leg 107 includes a distal opening 123 at a distal end 125 of distal artery leg 107.

As used herein, the proximal end of a prosthesis such as trifurcated modular stent device 100 is the end closest to the heart via the path of blood flow whereas the distal end is the end furthest away from the heart during deployment. In contrast and of note, the distal end of the catheter is usually identified to the end that is farthest from the operator/handle while the proximal end of the catheter is the end nearest the operator/handle.

For purposes of clarity of discussion, as used herein, the distal end of the catheter is the end that is farthest from the operator (the end furthest from the handle) while the distal end of trifurcated modular stent device 100 is the end nearest the operator (the end nearest the handle), i.e., the distal end of the catheter and the proximal end of trifurcated modular stent device 100 are the ends furthest from the handle while the proximal end of the catheter and the distal end of trifurcated modular stent device 100 are the ends nearest the handle. However, those of skill in the art will understand that depending upon the access location, trifurcated modular stent device 100 and the delivery system descriptions may be consistent or opposite in actual usage.

Main body 102 includes graft material 126 and one or more circumferential stents 128 coupled to graft material 126. Graft material 126 may be any suitable graft material, for example and not limited to, woven polyester, DACRON® material, expanded polytetrafluoroethylene, polyurethane, silicone, electro spun materials, or other suitable materials.

Circumferential stents 128 may be coupled to graft material 126 using stitching or other means. In the embodiment shown in FIGS. 1 and 2, circumferential stents 128 are coupled to an outside surface of graft material 126. However, circumferential stents 128 may alternatively be coupled to an inside surface of graft material 126.

Although shown with a particular number of circumferential stents 128, in light of this disclosure, those of skill in the art will understand that main body 102 may include a greater or smaller number of stents 128, e.g., depending upon the desired length of main body 102 and/or the intended application thereof.

Circumferential stents 128 may be any stent material or configuration. As shown, circumferential stents 128, e.g., self-expanding members, are preferably made from a shape memory material, such as nickel-titanium alloy (nitinol), and are formed into a zig-zag configuration. The configuration of circumferential stents 128 is merely exemplary, and circumferential stents 128 may have any suitable configuration, including but not limiting to a continuous or non-continuous helical configuration. In another embodiment, circumferential stents 128 are balloon expandable stents.

Further, main body 102 includes a longitudinal axis LA1. A lumen 130 is defined by graft material 126, and generally by main body 102. Lumen 130 extends generally parallel to longitudinal axis LA1 and between proximal opening 108 and distal end 112 of main body 102. Graft material 126 is cylindrical having a substantially uniform diameter in this embodiment. However, in other embodiments, graft material 126 varies in diameter, e.g., tapers or flares.

Bypass gate 104 includes graft material 132 and one or more circumferential stents 134 coupled to graft material 132. Graft material 132 may be any suitable graft material such as that described above regarding graft material 126. Circumferential stents 134 may be any stent material or configuration such at that described above regarding circumferential stents 128.

Circumferential stents 134 may be coupled to graft material 132 using stitching or other means. In the embodiment shown in FIGS. 1 and 2, circumferential stents 134 are coupled to an outside surface of graft material 132. However, circumferential stents 134 may alternatively be coupled to an inside surface of graft material 132.

Although shown with a particular number of circumferential stents 134, in light of this disclosure, those of skill in the art will understand that bypass gate 104 may include a greater or smaller number of stents 134, e.g., depending upon the desired length of bypass gate 104 and/or the intended application thereof.

Further, bypass gate 104 includes a longitudinal axis LA2. A lumen 136 is defined by graft material 132, and generally by bypass gate 104. Lumen 136 extends generally parallel to longitudinal axis LA2 and between proximal end 114 and distal opening 118 of bypass gate 104. Graft material 132 is cylindrical having a substantially uniform diameter in this embodiment. However, in other embodiments, graft material 132 varies in diameter, e.g., tapers or flares.

Primary artery leg 106 includes graft material 138 and one or more circumferential stents 140 coupled to graft material 138. Graft material 138 may be any suitable graft material such as that described above regarding graft material 126. Circumferential stents 140 may be any stent material or configuration such at that described above regarding circumferential stents 128.

Circumferential stents 140 may be coupled to graft material 138 using stitching or other means. In the embodiment shown in FIGS. 1 and 2, circumferential stents 140 are coupled to an outside surface of graft material 138. However, circumferential stents 140 may alternatively be coupled to an inside surface of graft material 138.

Although shown with a particular number of circumferential stents 140, in light of this disclosure, those of skill in the art will understand that primary artery leg 106 may include a greater or smaller number of stents 140, e.g., depending upon the desired length of primary artery leg 106 and/or the intended application thereof.

Further, primary artery leg 106 includes a longitudinal axis LA3. A lumen 142 is defined by graft material 138, and generally by primary artery leg 106. Lumen 142 extends generally parallel to longitudinal axis LA3 and between proximal end 116 and distal opening 122 of primary artery leg 106. Graft material 138 is cylindrical having a substantially uniform diameter in this embodiment. However, in other embodiments, graft material 138 varies in diameter, e.g., tapers or flares.

Distal artery leg 107 includes graft material 139 and one or more circumferential stents 141 coupled to graft material 139. Graft material 139 may be any suitable graft material such as that described above regarding graft material 126. Circumferential stents 141 may be any stent material or configuration such at that described above regarding circumferential stents 128.

Circumferential stents 141 may be coupled to graft material 139 using stitching or other means. In the embodiment shown in FIGS. 1 and 2, circumferential stents 141 are coupled to an outside surface of graft material 139. However, circumferential stents 141 may alternatively be coupled to an inside surface of graft material 139.

Although shown with a particular number of circumferential stents 141, in light of this disclosure, those of skill in the art will understand that distal artery leg 107 may include a greater or smaller number of stents 141, e.g., depending upon the desired length of distal artery leg 107 and/or the intended application thereof.

Further, distal artery leg 107 includes a longitudinal axis LA4. A lumen 143 is defined by graft material 139, and generally by distal artery leg 107. Lumen 143 extends generally parallel to longitudinal axis LA4 and between proximal end 117 and distal opening 123 of distal artery leg 107. Graft material 139 is cylindrical having a substantially uniform diameter in this embodiment. However, in other embodiments, graft material 139 varies in diameter, e.g., tapers or flares.

Generally, main body 102 is trifurcated at distal end 112 into bypass gate 104, primary artery leg 106, and distal artery leg 107. More particularly, lumen 130 of main body 102 is trifurcated into lumen 136 of bypass gate 104, lumen 142 of primary artery leg 106, and lumen 143 of distal artery leg 107.

In one embodiment, graft materials 126, 132, 138, 139 may be the same graft material, e.g., may be a single piece of graft material cut and sewn. However, in other embodiments, one or more of graft materials 126, 132, 138, 139 may be different that the others of graft materials 126, 132, 138, 139, e.g., different graft materials are cut and sewn together.

In the relaxed configuration of trifurcated modular stent device 100 as illustrated in FIGS. 1, 2, and 3, longitudinal axes LA1, LA2, LA3, and LA4 are parallel with one another such that bypass gate 104, primary artery leg 106, and distal artery leg 107 extend distally from main body 102.

Main body 102 has a first diameter D1, bypass gate 104 has a second diameter D2, primary artery leg 106 has a third diameter D3, and distal artery leg 107 has a fourth diameter D4. In accordance with this embodiment, first diameter D1 is greater than second diameter D2. Further, second diameter D2 is greater than third diameter D3 and fourth diameter D4. Third diameter D3 is equal to fourth diameter D4 in one embodiment. In other embodiments, third diameter D3 is greater or less than fourth diameter D4, e.g., depending upon the branch vessels to be perfused.

In accordance with this embodiment, first diameter D1 is greater than second diameter D2 combined with third diameter D3 and fourth diameter D4 (D1>(D2+D3+D4)) such that bypass gate 104, primary artery leg 106, and distal artery leg 107 are located within an imaginary cylinder defined by graft material 126 of main body 102 extended in the distal direction. The parallel design mimics anatomical blood vessel trifurcations to limit flow disruptions.

In one embodiment, first diameter D1 is greater than second diameter D2 combined with third diameter D3 and fourth diameter D4 (D1>(D2+D3+D4)) at distal end 112 and proximal ends 114, 116, 117, sometimes called the transition region. However, main body 102, bypass gate 104, primary artery leg 106, and/or distal artery leg 107 flare or taper away from the transition region in accordance with another embodiment, so D1>(D2+D3+D4) at the transition region but is not necessarily correct in regions away from the transition region. Flaring is indicated by the dashed lines in FIG. 2.

Stated another way, the transition region from main body 102 to bypass gate 104, primary artery leg 106, and distal artery leg 107 does not exceed first diameter D1 of main body 102. This insures bypass gate 104, primary artery leg 106, and distal artery leg 107 don't crush each other or negatively impact flow in any way. By avoiding having bypass gate 104, primary artery leg 106 and distal artery leg 107 extend out wider than main body 102, a good seal of stents 128 of main body 102 against the aorta is insured and type I endoleaks are minimized or avoided.

In accordance with one embodiment, the transition region between main body 102 and bypass gate 104, primary artery leg 106 and distal artery leg 107 is fully supported by one or more supporting stents, e.g., stents 128, 134, 140, 141, to prevent kinking in angled anatomy. Absent the supporting stents, trifurcated modular stent device 100 may be predispose to kinking in type III arches or gothic arches.

Main body 102 has a first length L1 in a direction parallel to the longitudinal axis LA1, bypass gate 104 has a second length L2 in a direction parallel to the longitudinal axis LA2, primary artery leg 106 has a third length L3 in a direction parallel to the longitudinal axis LA3, and distal artery leg 107 has a fourth length L4 in a direction parallel to the longitudinal axis LA4. In accordance with this embodiment, third length L3 is greater than second length L2 and fourth length L4 such that distal opening 122 the primary artery leg 106 is distal to distal opening 118 of bypass gate 104 and distal opening 123 of distal artery leg 107. Generally, primary artery leg 106 is longer than bypass gate 104 and distal artery leg 107.

Although fixed diameters D1, D2, D3, and D4 are illustrated and discussed, in one embodiment, main body 102, bypass gate 104, primary artery leg 106, and/or distal artery leg 107 are non-uniform in diameter. For example, main body 102 flares or tapers at proximal end 110. Similarly, bypass gate 104, primary artery leg 106, and/or distal artery leg 107 flare or taper at distal ends 120, 124, 125, respectively. For example, bypass gate 104, primary artery leg 106 and/or distal artery leg 107 flare or taper at distal ends 120, 124, 125 to enhance sealing.

Primary artery leg 106 and distal artery leg 107 are configured to exert a higher radial force than the radial force of bypass gate 104. As used herein, “radial force” includes both a radial force exerted during expansion/deployment as well as a chronic radial force continuously exerted after implantation such that a scaffold has a predetermined compliance or resistance as the surrounding native anatomy, e.g., the aorta, expands and contracts during the cardiac cycle. The radial force of bypass gate 104 is configured to be lower than that of primary artery leg 106 and distal artery leg 107 to avoid collapse of primary artery leg 106 and distal artery leg 107 when bypass gate 104 is deployed against and adjacent thereof and thus maintain perfusion of the brachiocephalic artery and an artery distal of the brachiocephalic artery, e.g., the left common carotid artery or the left subclavian artery, as discussed further below.

To configure bypass gate 104 and primary artery leg 106, distal artery leg 107 with differing relative radial forces, circumferential stents 140, 141 of primary artery leg 106, distal artery leg 107, respectively, are constructed with relatively thicker and/or shorter segments of material than circumferential stents 134 of bypass gate 104. Shorter and/or thicker circumferential stents 140, 141 have less flexibility but greater radial force to ensure that circumferential stents 134 of bypass gate 104 do not collapse lumens 142, 143 of primary artery leg 106, distal artery leg 107, respectively. Other variations or modification of circumferential stents 134, 140, 141 may be used to achieve relative radial forces in other embodiments.

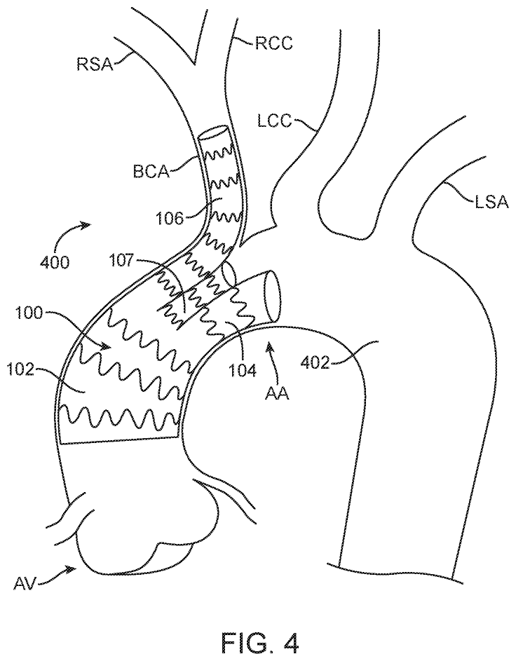

FIG. 4 is a cross-sectional view of a vessel assembly 400 including trifurcated modular stent device 100 of FIGS. 1, 2, and 3 after deployment in accordance with one embodiment. Referring to FIGS. 1-4 together, the thoracic aorta 402 has numerous arterial branches. The arch AA of the aorta 402 has three major branches extending therefrom, all of which usually arise from the convex upper surface of the arch AA. The brachiocephalic artery BCA originates anterior to the trachea. The brachiocephalic artery BCA divides into two branches, the right subclavian artery RSA (which supplies blood to the right arm) and the right common carotid artery RCC (which supplies blood to the right side of the head and neck). The left common carotid artery LCC artery arises from the arch AA of the aorta 402 just distal of the origin of the brachiocephalic artery BCA. The left common carotid artery LCC supplies blood to the left side of the head and neck. The third branch arising from the aortic arch AA, the left subclavian artery LSA, originates behind and just to the left of the origin of the left common carotid artery LCC and supplies blood to the left arm.

However, a significant proportion of the population has only two great branch vessels coming off the aortic arch AA while others have four great branch vessels coming of the aortic arch AA. Accordingly, although a particular anatomical geometry of the aortic arch AA is illustrated and discussed, in light of this disclosure, those of skill in the art will understand that the geometry of the aortic arch AA has anatomical variations and that the various structures as disclosed herein would be modified accordingly.

Aneurysms, dissections, penetrating ulcers, intramural hematomas and/or transections, generally referred to as a diseased region of the aorta 402, may occur in the aorta arch AA and the peripheral arteries BCA, LCC, LSA. For example, thoracic aortic aneurysms include aneurysms present in the ascending thoracic aorta, the aortic arch AA, and one or more of the branch arteries BCA, LCC, LSA that emanate therefrom. Thoracic aortic aneurysms also include aneurysms present in the descending thoracic aorta and branch arteries that emanate therefrom. Accordingly, the aorta 402 as illustrated in FIG. 4 has a diseased region similar to any one of those discussed above which will be bypassed and excluded using trifurcated modular stent device 100 as discussed below.

To deploy trifurcated modular stent device 100, a guide wire is introduced via supra aortic access, e.g. through the right subclavian artery RSA and the brachiocephalic artery BCA, and advanced into the ascending aorta 402. A delivery system including trifurcated modular stent device 100 is introduced via supra aortic access, e.g. through the right subclavian artery RSA and the brachiocephalic artery BCA, and is advanced into the ascending aorta 402 over the guidewire. The delivery system is positioned at the desired location such that the position of trifurcated modular stent device 100 is in the ascending aorta near the aortic valve AV.

Once positioned, a delivery sheath of the delivery system is withdrawn to expose main body 102, bypass gate 104, primary artery leg 106, and distal artery leg 107. This deploys trifurcated modular stent device 100.

More particularly, primary artery leg 106 self-expands (or is balloon expanded) into the brachiocephalic artery BCA. Main body 102, bypass gate 104, and distal artery leg 107 self-expand (or are balloon expanded) into the aorta 402.

As primary artery leg 106, distal artery leg 107 have a greater radial force than bypass gate 104, primary artery leg 106, distal artery leg 107 remains un-collapsed and opened. Accordingly, blood flow through primary artery leg 106 and perfusion of the brachiocephalic artery BCA and preservation of blood flow to cerebral territories including the brain is insured. This avoids stroke, or other medical complications from occlusion of the brachiocephalic artery BCA.

Perfusion of the brachiocephalic artery BCA is immediate and dependable. More particularly, primary artery leg 106 is released within brachiocephalic artery BCA and accordingly is necessarily located therein. Primary artery leg 106 is located within brachiocephalic artery BCA regardless of the radial orientation or longitudinal (axial) placement of trifurcated modular stent device 100 within the aorta 402. By avoiding the requirement of precise radial orientation and longitudinal placement of trifurcated modular stent device 100, the complexity of the procedure of deploying trifurcated modular stent device 100 is reduced thus insuring the most possible favorable outcome.

If there is any collapse between primary artery leg 106, distal artery leg 107 and bypass gate 104, the collapse is in bypass gate 104. However, bypass gate 104 has a sufficiently large diameter such that any collapse of bypass gate 104 is partial and blood flow through bypass gate 104 and the aorta 402 is maintained. Bypass gate 104 is opened thus insuring perfusion to distal territories, e.g., including the aorta 402, the left common carotid LCC, and the left subclavian artery LCA.

The design of bypass gate 104 limits wind socking of trifurcated modular stent device 100 during deployment. More particularly, the relatively large diameter D2 of bypass gate 104 readily allows blood flow through bypass gate 104 thus minimizing undesirable motion of trifurcated modular stent device 100 during deployment.

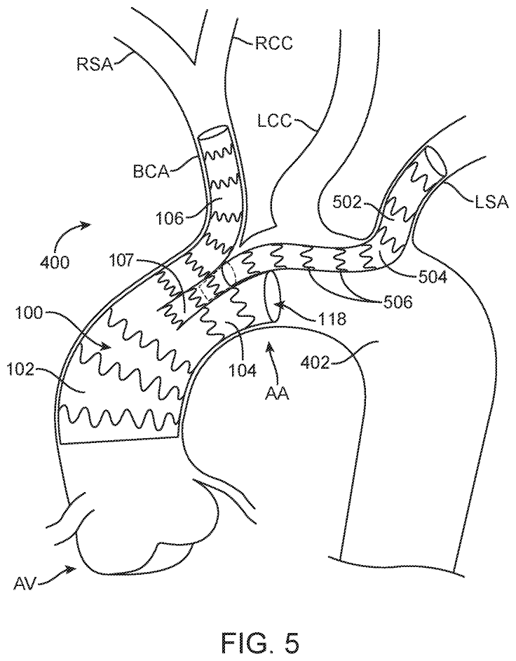

FIG. 5 is a cross-sectional view of vessel assembly 400 of FIG. 4 at a later stage after deployment of a bridging stent graft 502, sometimes called a bridging stent, in accordance with one embodiment. Referring now to FIG. 5, bridging stent graft 502 is located within distal artery leg 107 and the left subclavian artery LSA. More particularly, bridging stent graft 502 self-expands (or is balloon expanded) to be anchored within distal artery leg 107 and the left subclavian artery LSA thus providing a bypass from distal artery leg 107 to the left subclavian artery LSA.

Bridging stent graft 502 includes graft material 504 and one or more circumferential stents 506. Graft material 504 may be any suitable graft material such as that described above regarding graft material 126. Circumferential stents 506 may be any stent material or configuration such at that described above regarding circumferential stents 128.

Upon deployment of bridging stent graft 502, blood flow into distal artery leg 107 is bridged and passed into the left subclavian artery LSA through bridging stent graft 502.

In one embodiment, bridging stent graft 502 is deployed via supra aortic access. For example, to deploy bridging stent graft 502, a guide wire is introduced through the left subclavian artery LSA, and advanced into distal artery leg 107.

A delivery system including bridging stent graft 502 is introduced via supra aortic access and is advanced into the left subclavian artery LSA and distal artery leg 107 over the guidewire. Bridging stent graft 502 is then deployed from the delivery system, e.g., by removal of a sheath constraining bridging stent graft 502.

In another embodiment, bridging stent graft 502 is deployed via femoral access. For example, to deploy bridging stent graft 502, a guide wire is introduced via femoral access, i.e., is inserted into the femoral artery and routed up and into distal opening 118 of bypass gate 104. The guidewire is then routed from bypass gate 104 through distal artery leg 107 and into the left subclavian artery LSA.

A delivery system including bridging stent graft 502 is introduced via femoral access and is advanced into distal artery leg 107 and the left subclavian artery LSA over the guidewire. Bridging stent graft 502 is then deployed from the delivery system, e.g., by removal of a sheath constraining bridging stent graft 502.

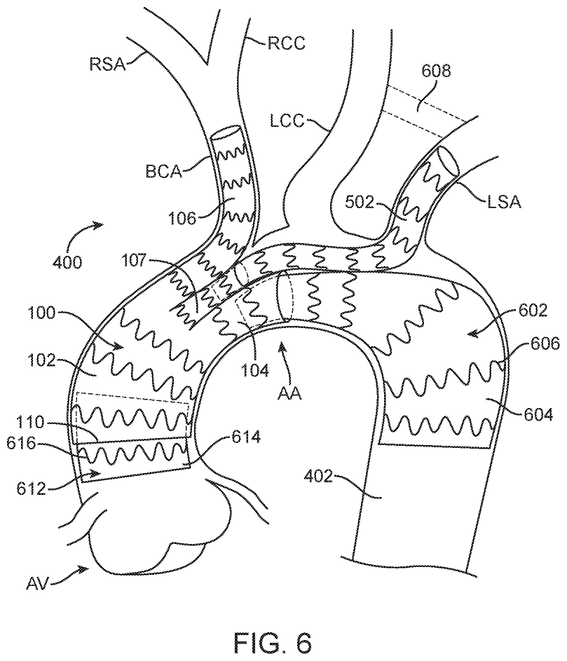

FIG. 6 is a cross-sectional view of vessel assembly 400 of FIG. 5 at a final stage after deployment of a tube graft 602 and a proximal cuff 612 into trifurcated modular stent device 100 in accordance with one embodiment. Referring to FIG. 6, tube graft 602 is deployed into bypass gate 104 and into aorta 402 and is attached thereto. For example, tube graft 602 extends distally beyond the ostium of the left subclavian artery LSA and seals in a more distal portion of the aorta 402, e.g., in healthy tissue.

Tube graft 602 includes graft material 604 and one or more circumferential stents 606. Graft material 604 may be any suitable graft material such as that described above regarding graft material 126. Circumferential stents 606 may be any stent material or configuration such at that described above regarding circumferential stents 128.

In accordance with this embodiment, tube graft 602 bypasses the left common carotid artery LCC. In accordance with this embodiment, a bypass 608, e.g., a surgically inserted bypass graft, provides perfusion to the left common carotid artery LCC. Illustratively, bypass 608 provides perfusion of the left common carotid artery LCC from the left subclavian artery LSA, e.g., provides a connection between the left common carotid artery LCC and the left subclavian artery LSA.

Bypass 608 is surgically inserted during the same procedure as deployment of trifurcated modular stent device 100 and tube graft 602. However, in another embodiment, bypass 608 is surgically inserted prior to deployment of trifurcated modular stent device 100 and tube graft 602, e.g., to simplify the procedure.

Further, as illustrated in FIG. 6, optionally, proximal cuff 612 is coupled to main body 102 of trifurcated modular stent device 100 and extend proximately therefrom. For example, proximal cuff 612 is deployed in the event that proximal end 110 of main body 102 is deployed distally from the aortic valve AV to extend between the desired deployment location and proximal end 110 of main body 102. Proximal cuff 612 is optional and in one embodiment is not deployed or used.

Proximal cuff 612 includes graft material 614 and one or more circumferential stents 616. Graft material 614 may be any suitable graft material such as that described above regarding graft material 126. Circumferential stents 616 may be any stent material or configuration such at that described above regarding circumferential stents 128.

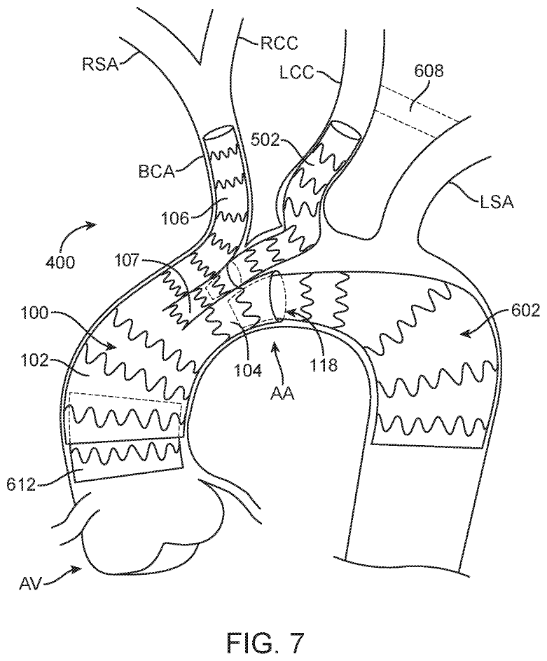

FIG. 7 is a cross-sectional view of vessel assembly 400 of FIG. 4 at a later stage after deployment of bridging stent graft 502 in accordance with another embodiment. Referring now to FIG. 7, bridging stent graft 502 is located within distal artery leg 107 and the left common carotid artery LCC. More particularly, bridging stent graft 502 self-expands (or is balloon expanded) to be anchored within distal artery leg 107 and the left common carotid artery LCC thus providing a bypass from distal artery leg 107 to the left common carotid artery LCC.

Upon deployment of bridging stent graft 502, blood flow into distal artery leg 107 is bridged and passed into the left common carotid artery LCC through bridging stent graft 502.

In one embodiment, bridging stent graft 502 is deployed via supra aortic access. For example, to deploy bridging stent graft 502, a guide wire is introduced through the left common carotid artery LCC, and advanced into distal artery leg 107.

A delivery system including bridging stent graft 502 is introduced via supra aortic access and is advanced into the left common carotid artery LCC and distal artery leg 107 over the guidewire. Bridging stent graft 502 is then deployed from the delivery system, e.g., by removal of a sheath constraining bridging stent graft 502.

In another embodiment, bridging stent graft 502 is deployed via femoral access. For example, to deploy bridging stent graft 502, a guide wire is introduced via femoral access, i.e., is inserted into the femoral artery and routed up and into distal opening 118 of bypass gate 104. The guidewire is then routed from bypass gate 104 through distal artery leg 107 and into the left common carotid artery LCC.

A delivery system including bridging stent graft 502 is introduced via femoral access and is advanced into distal artery leg 107 and the left common carotid artery LCC over the guidewire. Bridging stent graft 502 is then deployed from the delivery system, e.g., by removal of a sheath constraining bridging stent graft 502.

Optionally, tube graft 602 and proximal cuff 612 are deployed as discussed above regarding FIG. 6. In accordance with this embodiment, tube graft 602 bypasses the left subclavian artery LSA. In accordance with this embodiment, bypass 608 provides perfusion to the left subclavian artery LSA. Illustratively, bypass 608 provides perfusion of the left subclavian artery LSA from the left common carotid artery LCC.

Generally, bridging stent graft 502 is deployed within the left subclavian artery LSA (FIG. 5) or the left common carotid artery LCC (FIG. 7) after sub selecting each gate.

This application is related to commonly assigned: U.S. patent application Ser. No. 16/367,889, filed on Mar. 28, 2019, entitled “MODULAR STENT DEVICE FOR MULTIPLE VESSELS AND METHOD”, of Perkins et al.; U.S. patent application Ser. No. 16/367,906, filed on Mar. 28, 2019, entitled “SUPRA AORTIC ACCESS MODULAR STENT ASSEMBLY AND METHOD”, of Perkins et al.; U.S. patent application Ser. No. 16/367,992, filed on Mar. 28, 2019, entitled “FEMORAL AORTIC ACCESS MODULAR STENT ASSEMBLY AND METHOD, of Perkins et al.; U.S. patent application Ser. No. 16/554,813, filed on Aug. 29, 2019, entitled “MODULAR MULTIBRANCH STENT ASSEMBLY AND METHOD”, of Perkins et al.; U.S. patent application Ser. No. 16/527,769, filed on Jul. 31, 2019, entitled “MODULAR MULTIBRANCH STENT ASSEMBLY AND METHOD”, of Perkins et al.; U.S. patent application Ser. No. 16/502,462, filed on Jul. 3, 2019, entitled “SINGLE MULTIBRANCH STENT DEVICE ASSEMBLY AND METHOD”, of Perkins et al.; and U.S. patent application Ser. No. 16/585,722, filed Sep. 27, 2019, entitled “DOCKING GRAFT FOR PLACEMENT OF PARALLEL DISTALLY EXTENDING GRAFTS ASSEMBLY AND METHOD”, of Perkins et al., which are herein incorporated by reference in their entireties.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

What is claimed is:

1. A method comprising:
introducing a delivery system including a trifurcated modular stent device via supra aortic access through a brachiocephalic artery;
advancing the delivery system into an aorta;
deploying the trifurcated modular stent device from the delivery system such that a main body of the trifurcated modular stent device engages the aorta, a primary artery leg of the trifurcated modular stent device engages the brachiocephalic artery, a bypass gate of the trifurcated modular stent device engages the aorta, and a distal artery leg of the trifurcated modular stent device is located within the aorta proximal of a distal artery distal of the brachiocephalic artery.

2. The method of claim 1 further comprising deploying a bridging stent graft within the distal artery leg and within the distal artery.

3. The method of claim 2 wherein the distal artery is selected from the group consisting of a left subclavian artery and a left common carotid artery.

4. The method of claim 1 wherein the primary artery leg and the distal artery leg partially collapse the bypass gate.

5. The method of claim 1 wherein the deploying comprises withdrawing a delivery sheath of the delivery system to expose the trifurcated modular stent device, the primary artery leg expanding into the brachiocephalic artery upon the exposing.

6. The method of claim 5 wherein perfusion of the brachiocephalic artery through the primary artery leg upon the expanding of the primary artery leg is immediate.

7. The method of claim 5 wherein the primary artery leg is located within the brachiocephalic artery regardless of a radial orientation or a longitudinal placement of the trifurcated modular stent device within the aorta.

8. The method of claim 5 wherein the main body, the bypass gate, and the distal artery leg expanding into the aorta upon the exposing.

9. The method of claim 2 wherein the deploying the bridging stent graft comprises advancing the bridging stent graft through the distal artery and into the distal artery leg.

10. The method of claim 2 wherein the deploying the bridging stent graft comprises advancing the bridging stent graft through the bypass gate, through the distal artery leg, and into the distal artery.

11. The method of claim 1 further comprising deploying a tube graft within the bypass gate and within the aorta, the tube graft extending distally from the bypass gate.

12. The method of claim 1 further comprising deploying a proximal cuff within the main body and within the aorta, the proximal cuff extending proximally from the main body.

13. The method of claim 1 wherein the main body is trifurcated into the bypass gate, the primary artery leg, and the distal artery leg at a single transition region.

14. A method comprising:
deploying a trifurcated modular stent device comprising:
locating a main body of the trifurcated modular stent device in an aorta;
locating a bypass gate of the trifurcated modular stent device in the aorta;
locating a primary artery leg of the trifurcated modular stent device within a brachiocephalic artery, a proximal end of the primary artery leg being directly coupled to the distal end of the main body; and
perfusing a distal artery distal of the brachiocephalic artery with a distal artery leg of the trifurcated modular stent device, a proximal end of the distal artery leg being directly coupled to the distal end of the main body,
wherein the main body is trifurcated into the bypass gate, the primary artery leg, and the distal artery leg at a single transition region, the bypass gate being directly adjacent to the primary artery leg and the distal artery leg at the transition region,
wherein a length of the primary artery leg is greater than a length of the bypass gate and a length of the distal artery leg,
wherein one or more of the main body, the bypass gate, the primary artery leg, and the distal artery leg flare away from the transition region.

15. The method of claim 14 further comprising locating a bridging stent graft within the distal artery leg and the distal artery.

16. The method of claim 14 wherein the length of the bypass gate is greater than the length of the distal artery leg.

17. A method comprising:
deploying a trifurcated modular stent device, the trifurcated modular stent device comprising:
a main body;
a bypass gate extending distally from a distal end of the main body;
a primary artery leg extending distally from the distal end of the main body, a proximal end of the primary artery leg being directly coupled to the distal end of the main body; and
a distal artery leg extending distally from the distal end of the main body, a proximal end of the distal artery leg being directly coupled to the distal end of the main body,
wherein the main body is trifurcated into the bypass gate, the primary artery leg, and the distal artery leg at a single transition region, the bypass gate being directly adjacent to the primary artery leg and the distal artery leg at the transition region,
wherein a length of the primary artery leg is greater than a length of the bypass gate and a length of the distal artery leg,
wherein one or more of the main body, the bypass gate, the primary artery leg, and the distal artery leg flare away from the transition region.

18. The method of claim 17 wherein the main body has a first longitudinal axis, the bypass gate has a second longitudinal axis, the primary artery leg has a third longitudinal axis, and the distal artery leg has a fourth longitudinal axis, the first, second, third, and fourth longitudinal axes are parallel with one another when the modular stent device is in a relaxed configuration.

19. The method of claim 17 wherein the main body has a first diameter, the bypass gate has a second diameter, the primary artery leg has a third diameter, and the distal artery leg has a fourth diameter, the first diameter being greater than the second, third, and fourth diameters together at the transition region where the main body meets the bypass gate, the primary artery leg, and the distal artery leg.

20. The method of claim 19 wherein the bypass gate, the primary artery leg, and the distal artery leg are located within an imaginary cylinder defined by the main body extended in the distal direction at the transition region.
